(19) United States
(12) Patent Application Publication
(10) Pub. No.: US 2009/0134357 A1
(43) Pub. Date: May 28, 2009

(54) ACRYLIC ACID, WATER-ABSORBENT POLYMER STRUCTURES BASED ON RENEWABLE RESOURCES AND METHOD FOR PRODUCING SAID STRUCTURES

(76) Inventors: Gunther Bub, Marl (DE); Jurgen Mosler, Castrop-Rauxel (DE); Andreas Sabbagh, Dulmen (DE); Franz-Felix Kuppinger, Marl (DE); Stefan Nordhoff, Recklinghausen (DE); Guido Stochniol, Haltern (DE); Jorg Sauer, Dulmen (DE); Udo Knippenberg, Marl (DE); Franck Furno, Dusseldorf (DE); Gunter Latoschinski, Marl (DE); Michael Bernd Fricke, Hochmoor (DE); Horst-Werner Zanthoff, Mulheim An Der Ruhr (DE)

Correspondence Address: SMITH MOORE LEATHERWOOD LLP, P.O. BOX 21927, GREENSBORO, NC, 27420, US

(21) Appl. No.: 11/817,247

(22) PCT Filed: Feb. 28, 2006
(86) PCT No.: PCT/EP2006/001831

§ 371 Date: Mar. 19, 2008

(30) Foreign Application Priority Data
Feb. 28, 2005 (DE) 10 2005 009 586.0

(51) Int. Cl. B01J 20/26; C07C 27/10; C08F 2/00; B01J 19/00

(52) U.S. Cl. 252/194; 562/512.2; 526/75; 422/189; 422/134

(57) ABSTRACT

The present invention relates to a process for production of acrylic acid, comprising at least the following steps:
a. dehydrating glycerine to a dehydration product comprising acrolein;
b. gas phase oxidation of the dehydration product comprising acrolein to obtain a monomer gas comprising an acrylic acid;
c. bringing into contact the monomer gas with a quench means to obtain a quench phase comprising acrylic acid; and
d. processing the quench phase comprising acrylic acid to obtain a monomer phase comprising acrylic acid.

The present invention may also relate to a process for preparation of polymers by radical polymerization of acrylic acid, the water-absorbing polymers obtainable by this process, water-absorbing polymers based to at least 25 wt % upon partially neutralized acrylic acid, a composite, a device for preparation of acrylic acid, a process for preparation of acrylic acid, and the acrylic acid obtainable by this process.

This application is a national stage application under 35 U.S.C. 371 of international application No. PCT/EP2006/001831 filed 28 Feb. 2006, and claims priority to German Application No. DE 10 2005 009 586.0 filed 28 Feb. 2005, the disclosure of which is expressly incorporated herein by reference.

### BACKGROUND

The present invention relates to a process for preparation of acrylic acid, a process for preparation of polymers by radical polymerization of acrylic acid, preferably for preparation of water-absorbing polymers, the water-absorbing polymers obtainable by this process, water-absorbing polymers based to at least 25 wt % upon partially neutralized acrylic acid, a composite, a process for producing a composite, the composite obtainable by this process, the use of acrylic acid in the preparation of water-absorbing polymer structures, a device for preparation of acrylic acid, a process for preparation of acrylic acid, and the acrylic acid obtainable by this process.

High requirements are made of the purity of acrylic acid which is used in the preparation of polymeric compounds. This is particularly the case when the polymers are so-called superabsorbent polymers, which are incorporated into wound dressings or hygiene articles. These polymers can absorb and thus bind aqueous liquids to form a hydrogel. Superabsorbent polymers are, therefore, used in particular in hygiene articles such as diapers, incontinence articles, sanitary napkins and the like for the absorption of body fluids. A comprehensive overview of superabsorbent polymers, their application and their preparation is given by F. L. Buchholz and A. T. Graham (Editors) in “Modern Superabsorbent Polymer Technology”, Wiley-VCH, New York, 1998.

In the preparation of superabsorbent polymers, generally an acrylic acid is used which has been obtained as pure acrylic acid by catalytic gas phase oxidation of propylene to acrolein, which is then converted in a further catalytic gas phase oxidation to acrylic acid, subsequent absorption of the gaseous reaction mixture in water, distillation of the thus-obtained aqueous acrylic acid solution to obtain a crude acrylic acid, and further purification of the crude acrylic acid by distillation or crystallization.

It is disadvantageous in this process for production of acrylic acid that the temperatures between 300° C. and 450° C. used in both stages lead to formation of oligomers and further undesired cracking products. This results in the accumulation of an undesirably large amount of compounds which are less volatile than acrylic acid or compounds which are difficult to separate from acrylic acid, such as acetic acid, which must be separated from the acrylic acid. This separation, which as a rule occurs by distillation, leads to a further thermal stress on the acrylic acid, which favors the disadvantageous formation of dimeric or oligomeric acrylic acid. A high acrylic acid dimer or acrylic acid oligomer content is, however, disadvantageous, since these dimers or oligomers are built into the polymer backbone during the preparation of superabsorbent polymers by radical polymerization of acrylic acid in the presence of crosslinkers. During the post-treatment of the surface of the polymer particles, which occurs following the polymerization, for example as a surface post-crosslinking, the polymerized-in dimers, however, split to form β-hydroxypropionic acid, which is dehydrated under the post-crosslinking conditions to form acrylic acid. A high dimeric acrylic acid content in the acrylic acid used in the production of superabsorber therefore leads to the acrylic acid monomer content increasing during a thermal treatment of the polymer, as occurs during post-crosslinking.

Since the soluble parts, in particular the acrylic acid monomers in the superabsorbent polymers, can cause skin irritation, a use of these polymers in hygiene articles requires a particularly low content in extractable components. Also other, often toxic compounds remain in the acrylic acid obtained by the catalytic gas phase oxidation. Among these impurities are included, in particular, aldehydes, which have a disruptive effect on the course of polymerization, with the result that the polymers still comprise considerable amounts of soluble components.

Acrylic acids produced in previous ways from propylene comprises not inconsiderable amounts of ketones having double bonds, in particular protoanemonin (PTA). This compound can, on contact with skin, cause signs of poisoning, such as, for example, reddening, itching, or blister formation. Superabsorbent polymers which comprise large amounts of PTA as soluble components are therefore of concern from a dermatological viewpoint. Furthermore, PTA disrupts the polymerization, as described in US-A-2002/0120085. This leads to the obtaining of superabsorbent polymers with less good absorption, transport, and retention properties for body fluids, so that when using superabsorbent polymers of this type in hygiene articles such as diapers or sanitary napkins, wearer comfort is worsened, for example by “leakage”.

Several processes have already been described in the state of the art, with which the content in the above-mentioned compounds, in particular of aldehydes, or PTA, in the acrylic acid obtained by gas phase oxidation of propylene can be reduced.

EP-A-0 574 260 suggests using, in the production of superabsorber, if possible, an acrylic acid which is characterized by a β-hydroxypropionic acid content of not more than 1000 ppm. Such an acrylic acid is obtained by distilling conventional acrylic acid as directly as possible before the polymerization.

DE-A-101 38 150 suggests, in order to reduce the amount of aldehyde in the acrylic acid, bringing this into contact with an aldehyde trapper, in order to convert the aldehydes into high-boiling compounds, which can then be separated by means of distillation.

Various methods have been proposed in the state of the art for the removal of PTA, such as the addition of a nitrous acid salt, of nitrogen oxide or of nitrobenzene (JP 81-41614) or the addition of one or more para-phenylene diamines (EP-A-567 207) to the acrylic acid.

The disadvantage of the above-described processes for reducing the amount of aldehydes and ketones in acrylic acid is, however, among others, that, in so far as the impurity content of the acrylic acid is not known exactly, these reagents must be used in excess for the purpose of as complete a removal as possible of impurities from the acrylic acid. On the one hand, reagents which are reactive to the acrylic acid must be added. The portion of these reagents which is not converted must then be removed again. Reagents which are not removed are comprised in the superabsorbent polymer obtained from such an acrylic acid as soluble components, which can come into contact with the skin of the hygiene article wearer when the superabsorbent polymers are used in hygiene articles. Furthermore, the processes known from the prior art for removal of aldehydes or ketones from acrylic acid only very seldom remove these impurities completely.

In addition to the disadvantages which are traced back to impurities in the acrylic acid used in the production of superabsorbent polymers, known superabsorbent polymers also have the disadvantage that, unless they at least partially comprise natural polymers, such as celluloses, they are hardly based upon renewable raw materials. While it is successful to produce many of the components used in hygiene articles, in particular in disposable diapers, from biological starting materials, replacement of the superabsorbent polymers based upon cross-linked polyacrylates by natural superabsorbent polymers, such as cross-linked, derivative starches or celluloses, is generally associated with significant losses in respect of the absorbent properties. This mostly leads to the necessity of using considerably more of the absorbents based upon natural polymers, simply in order to approach the same absorbent properties in a hygiene article. This is disadvantageous, because the hygiene articles become more voluminous and heavier, which significantly restricts wearing comfort and leads to a larger waste volume, which, in addition to dumping space or combustion expenditure, also requires greater transport capacity for the removal of waste. All of this has a disadvantageous effect upon the environmental friendliness of the absorbers based upon natural polymers.

The object of the present invention was to overcome the disadvantages arising from the state of the art.

### SUMMARY

In particular, the present invention had the object of making available polymers, in particular superabsorbent polymers, which have a particularly low content in extractable, possibly toxic components.

Furthermore, another object of the present invention was to provide polymers, in particular superabsorbent polymers, which are environmentally friendly and still have excellent application properties. In particular it was desired to provide superabsorbent polymers with improved environmental friendliness while retaining the same good absorbent properties.

In addition, it was another object of the present invention to improve the environmental friendliness of further processing products comprising the polymers according to the invention, such as composites in general and hygiene articles in particular, without the desired functions, such as absorbent capability, wearing comfort, and simple producibility of these further processing products suffering.

It is another object of the present invention to provide a process for preparing polymers of this type and the monomers suitable for their production, whereby this process should take place as far as possible without the use of reactive compounds for removal of impurities from the monomers used in the preparation of the polymers.

Numerous other features and advantages of the present invention will appear from the following description. In the description, reference is made to exemplary embodiments of the invention. Such embodiments do not represent the full scope of the invention. Reference should therefore be made to the claims herein for interpreting the full scope of the invention. In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

In addition, another object of the present invention was to suggest a process and a device for the production of monomers and polymers, which can be integrated with as little conversion expenditure as possible into existing industrial manufacturing processes and devices.

### FIGURES

The foregoing and other features, aspects, and advantages of the present invention will become better understood with regard to the following description, appended claims, and accompanying drawings where:

FIG. 1 shows a schematic of the individual stages and steps of the process according to the invention and of the device according to the invention.

FIG. 2 shows a gas phase dehydration unit.

FIG. 3 shows a liquid phase dehydration unit.

FIG. 4 shows a processing unit.

FIG. 5 shows a further embodiment of the liquid phase dehydration unit.

### DETAILED DESCRIPTION

A contribution to the solution of the above mentioned objects is provided by a process for preparation of acrylic acid, comprising at least the following steps:
a. dehydrating glycerine to an acrolein-comprising dehydration product;
b. gas phase oxidation of the acrolein-comprising dehydration product to obtain an acrylic acid-comprising monomer gas;
c. bringing into contact the monomer gas with a quenching means to obtain an acrylic acid-comprising quench phase; and
d. processing of the quench phase to obtain an acrylic acid-comprising monomer phase.

A further contribution to the solution of the above objects is provided by a process for preparation of a polymer by radical polymerization of acrylic acid comprising at least the following steps:
A. dehydrating glycerine to an acrolein-comprising dehydration product;
B. Gas phase oxidation of the acrolein-comprising dehydration product to obtain an acrylic acid-comprising monomer gas;
C. bringing into contact the monomer gas with a quench means to obtain an acrylic acid-comprising quench phase;
D. processing the quench phase to obtain an acrylic acid-comprising monomer phase; and
E. polymerizing the monomer phase.

In one aspect of the process according to the invention, it is preferred that the glycerine is obtained by saponification of fats. These fats can be animal fats as well as vegetable fats. Animal fats are obtained in particular from the processing of animals. Vegetable fats are obtained in large amounts from oil extraction from oily fruits such as rape, soya, sesame, olives, and sunflower seeds. Large amounts of glycerine are generated, in particular, by the production of so-called “Bio diesel” from rape seed oil, as known from WO-A-2004/029016, among others. It is thus preferred in the process according to the invention that the glycerine is generated in the production of liquid fuels from natural raw materials. This is given in particular for saponification devices following oil mills.

In one aspect of the process according to an embodiment of the present invention, the dehydration occurs at least partially in the liquid phase. As the liquid phase, aqueous systems are particularly preferred. If the dehydration should be carried out at least partially or completely in a liquid phase, this has the advantage, in particular if this is an aqueous phase, that for high glycerine concentrations, high acrolein concentrations in the aqueous phase can be achieved. These aqueous phases with high acrolein concentrations can be used directly in the next stage of the gas phase oxidation. In order to protect the oxidation catalyst from carbonization, it is preferred to provide a separating unit between dehydration and oxidation. In this separating unit, the accompanying materials which are different from acrolein and the acrolein are conducted to the gas phase oxidation. In this way, significantly longer catalyst lifetimes can be achieved.

A further advantage of the liquid phase dehydration is that a rinsing effect can be achieved with the liquid phase, with which a formation of coating on a solid state catalyst can be significantly reduced, which leads to increased catalyst running times and thus to a reduced need for regeneration of the catalyst. A further advantage of the liquid phase dehydration is that it can be carried out at relatively moderate temperatures, within a range from about 160° C. to about 270° C., or within a range from about 180° C. to about 260° C., or within a range from about 215° C. to about 245° C. These temperature ranges lie significantly below the decomposition and boiling temperature of glycerine of about 290° C., which leads to reduction of sump and cracking products as well as other impurities, which have a disadvantageous effect on the operating time of the gas phase oxidation.

It is, however, provided in a further embodiment of the process according to the invention that the dehydration is carried out near to the decomposition point of glycerine, in order to increase the yield. In this embodiment, the temperatures are within a range from about 170° C. to about 290° C., or within a range from about 190° C. to about 289° C., or within a range from about 225° C. to about 288° C. It is preferred that the liquid phase dehydration occurs in a circular operation mode, in which, in the case of a solid state catalyst, the glycerine-containing liquid phase is conducted by means of a pump over the catalyst in a pressurized system. With liquid catalysis or homogeneous catalysis, at least a partial flow from the reactor is conducted in the cycle. Converted glycerine, consumed catalyst, and optionally removed water are added to the partial flow which has been fed back into the reactor during the cycle, preferably at the reactor entry. Should the partial flow be obtained from an acidic homogeneously catalyzed dehydration, it is advantageous that the partial flow is at least partially neutralized. By this measure, the formation of side products by reactions in the partial flow can be repressed or even completely prevented.

It is also advantageous to deplete materials with higher boiling points than acrolein, characterized as high boiling, from the partial flow. This can occur, for example by means of separating units comprising membranes, which membranes are preferably semi-permeable. In this way, higher turnovers and significantly fewer side-products can be obtained, in addition to higher selectivity, in a gentle way.

In a further embodiment of the process according to the invention, the dehydration occurs at least partially or also fully in a gas phase. The dehydration in the gas phase has proven particularly useful in the conversion of glycerine from saponification of fat and from the production of biodiesel. This glycerine generally has a high salt load, which can be separated very well by the evaporation step of the gas phase dehydration. As for the liquid phase dehydration, it is also preferred that the gas phase dehydration occurs in the presence of water.

Accordingly, it is preferred in the process according to the invention that the glycerine is used in an aqueous phase. In the case of the liquid phase dehydration, this liquid glycerine phase generally has a water content within a range from 0 wt % to about 97 wt %, for example from 0 wt % to about 30 wt %, or within a range from about 60 wt % to about 95 wt %, or within a range from about 70 wt % to about 90 wt %, whereby, however, a smaller water content, for example a water content within a range from 0 to about 20 wt % and from 0 wt % to about 10 wt % of water, respectively based upon the aqueous phase, is also conceivable.

In the case of a gas phase dehydration, the aqueous glycerine phase generally has a water amount within a range from 0 wt % to about 97 wt %, or within a range from about 60 wt % to about 95 wt %, or within a range from about 70 wt % to about 90 wt %, respectively based upon the aqueous glycerine phase, whereby it can also be advantageous here to use smaller amounts of water, for example a water content within a range from 0 wt % to about 20 wt % or from 0 wt % to about 10 wt %. The further principal component of the glycerine phase is glycerine. Further advantages of the gas phase dehydration are high turnovers up to a quantitative yield with, at the same time, high space-time-yields.

According to another embodiment of the process according to the invention, one can combine gas phase dehydration and liquid phase dehydration with each other. According to one form of the process according to the invention, the glycerine can first be conducted to the gas phase dehydration and then to the liquid phase dehydration or the other way around. The first-mentioned order has the advantage that glycerine charges with heavy salt loads originating from fat saponification can first be freed from these salt loads by evaporation in the gas phase dehydration, in order to be then further converted in the liquid phase dehydration by means of the cycle to high yields and selectivities with few side-products.

According to a further embodiment of the process according to the invention, a dehydration catalyst is used in the process. Dehydration catalysts can be acidic as well as basic catalysts. Acidic catalysts have a low tendency to form oligomers. The dehydration catalyst can be used as a homogeneous as well as a heterogeneous catalyst. If the dehydration catalyst is present as a heterogeneous catalyst, it is preferred that the dehydration catalyst is in contact with a carrier x. Carrier x may include and be all solids that appear suitable to the skilled person. In this context, it is preferred that the solid has suitable pore volumes, which are suited for a good binding and taking up of the dehydration catalyst. In addition, total pore volumes according to DIN 66133 are within a range from about 0.01 ml/g to about 3 ml/g, or within a range from about 0.1 ml/g to about 1.5 ml/g.

In addition, it is preferred that the solids suitable as carrier x. have a surface area within the range from about 0.001 m²/g to about 1000 m²/g, or from about 0.005 m²/g to about 450 m²/g, or from 0.01 m²/g to 300 m²/g according to BET test according to DIN 66131. A bulk good, which has an average particle diameter within the range from 0.1 mm to 40 mm, or from 1 mm to 10 mm, or from about 1.5 mm to about 5 mm, can be used as carrier for the dehydration catalyst. The wall of the dehydration reactor can also serve as carrier. Furthermore, the carrier can itself be acidic or basic, or an acidic or basic dehydration catalyst can be applied to an inert carrier. Application techniques may include immersion, or impregnation, or the incorporation into a carrier matrix.

Particularly suited as carrier x, which can also have dehydration catalyst properties, are natural or synthetic silicate materials, such as mordenite, montmorillonite, acidic zeolites, carrier materials supporting mono-, di- or polybasic inorganic acids, in particular phosphoric acids, or acidic salts of inorganic acids, such as oxidic or silicate materials, for example Al₂O₃, TiO₂, oxides, and mixed oxides, such as gamma-Al₂O₃ and ZnO—Al₂O₃ as well as Cu—Al mixed oxides of heteropolyacids.

According to an embodiment of the present invention, the carrier x consists at least partially of an oxidic compound. Such oxidic compounds should have at least one of the elements Si, Ti, Zr, Al, P, or a combination of at least two thereof. Such carriers can also function as dehydration catalysts through their acidic or basic properties. A class of compounds that function both as carrier x and as dehydration catalyst comprises silicon-aluminium-phosphorus oxides. Basic materials which function as both dehydration catalyst and as carrier x comprise alkali, alkaline earth, lanthanum, lanthanide, or a combination of at least two thereof in their oxidic form. Such oxidic or basic dehydration catalysts are commercially obtainable from Degussa AG and from Südchemie AG. Ion exchangers represent a further class. These may also be present in both basic and acidic form.

Examples of homogeneous dehydration catalysts may include inorganic acids, or phosphorous-comprising acids, or phosphoric acids, wherein these inorganic acids may be immobilized on the carrier x by immersion or impregnation.

The use of heterogeneous catalysts may be successfully used, in particular in gas phase dehydration. In liquid phase dehydration, however, both homogeneous and heterogeneous dehydration catalysts may be used.

In another embodiment of the inventive process, the dehydration catalyst may be an inorganic acid. The term acid is understood to mean in this text materials that behave according to the Brönsted definition of acids. All acids known to the skilled person and appearing suitable may be considered. Such acids may include S- and P-comprising acids, whereby the P-comprising acids may be used. It is preferred in connection with the liquid phase dehydration that this dehydration catalyst, which is also characterized as homogeneous, is present in a solution. This solution may be the aqueous glycerine-comprising phase used for dehydration. These homogeneous catalysts may be used in an amount within a range from about 0.0001 wt % to 20 wt %, or from about 0.001 wt % to about 15 wt %, or from about 0.01 wt % to about 10 wt %, respectively based on the phase to be dehydrated.

In addition, a dehydration catalyst may be used with an H₀ value within a range from +1 to −10, or from +2 to −8.2, or in the liquid phase dehydration within a range from +2 to −3 and in the gas phase dehydration within a range from −3 to −8.2. The H₀ value corresponds to the acidic function according to Hammett and may be determined by the so-called amine titration and use of indicators or by absorption of a gaseous base—see “Studies in Surface Science and Catalytics”, vol. 51, 1989: “New Solid Acids and Bases, their catalytic Properties”, K. Tannabe et. al. Further details on the production of acrolein from glycerine can also be found in DE 42 38 493 C1.

In another embodiment of the process according to the invention, the gas phase oxidation in step b) of the process may occur in the presence of one or more oxidation catalysts, which comprise transition metals in elemental or in chemically bound form or both. The oxidation catalysts may comprise at least one of the elements molybdenum, tungsten, vanadium, or a combination of at least two thereof in at least partially oxidized form. Oxidation catalysts of this sort may be used as heterogeneous catalysts in contact with a carrier y wherein the oxidation catalysts may be incorporated into this carrier y. As suitable carrier y, the compounds mentioned in connection with carrier x may be considered, whereby carriers based upon silicon oxide, or aluminium oxide, or aluminium-silicon oxide may be used. Oxidation catalysts of this type are widely described in the literature. Reference is here made, for example, to DE-A-26 26 887, EP-A-0 534 294, and to US-A-2002/0198406. Oxidation catalysts of this type for the conversion of acrolein to acrylic acid are commercially obtainable, for example from Nippon Kayaku KK and from Degussa AG.

The dehydration product of the present invention, optionally in an aqueous phase, may be conducted to the gas phase oxidation. The dehydration product may comprise at least about 10 wt %, or at least about 20 wt %, or at least about 40 wt % acrolein. The amount of water may lie within the range from about 0.001 wt % to about 50 wt %, or from about 0.1 wt % to about 50 wt %, or from about 10 wt % to about 40 wt %, or from about 12 wt % to 20 wt %, whereby these and the above wt % values are based respectively upon the phases fed into the gas phase oxidation.

Another embodiment of the process according to the invention provides that glycerine may be introduced into a dehydration space as an aqueous glycerine phase with a glycerine concentration within a range from about 0.1 wt % to about 90 wt %, or from about 1 wt % to about 80 wt %, or from 2 wt % to 50 wt %, respectively based upon the aqueous glycerine phase. This dehydration space comprises both a fluid phase and a gas phase, whereby the concentration of glycerine in the fluid phase may be greater than in the gas phase and the concentration of acrolein in the gas phase may be greater than in the fluid phase. The concentration of glycerine in the fluid phase may be greater than in the gas phase at least by a factor of about 1.1, or at least by a factor of about 2, or at least by a factor of about 5. The concentration of acrolein in the gas phase may be greater than in the fluid phase at least by a factor of about 1.1, or at least by a factor of about 2, or at least by a factor of about 5. In this embodiment, the acrolein may comprised in the gas phase is removed from the dehydration space, which may be designed as a pressure reactor. In connection with this embodiment, more dehydration of glycerine to acrolein may occur in the fluid phase than in the gas phase. More dehydration of glycerine to acrolein may occur in the fluid phase than in the gas phase by a factor of about 1.1, or at least by a factor of about 2, or at least by a factor of about 5. The acrolein formed in the fluid phase may be transferred into the gas phase. The volume of the dehydration space taken up by the fluid phase may be larger than the volume taken up by the gas phase. The volume of the dehydration space taken up by the fluid phase may be larger than the volume taken up by the gas phase, by a factor of about 1.1, or at least by a factor of about 2, or at least by a factor of about 5. The factors used here may be determined, for example, on the basis of investigations of the phase equilibria.

Another embodiment of the process according to the invention for production of acrylic acid may comprise the following process steps:
a. dehydrating glycerine in the form of an aqueous glycerine solution to a dehydration product comprising acrolein in the form of an aqueous acrolein solution by means of liquid phase dehydration with at least partial transfer of the aqueous acrolein solution into the gas phase, whereby the weight ratio of acrolein to glycerine in the gas phase is greater than the weight ratio of acrolein to glycerine in the aqueous acrolein solution by a factor of at least about 2, or at least about 4, or at least about 10, or at least about 100;
b. gas phase oxidation of the acrolein in the vapor phase to obtain a monomer gas comprising acrylic acid;
c. bringing into contact the monomer gas with a quench means to obtain a quench phase comprising acrylic acid; and
d. processing the quench phase to obtain a monomer phase comprising acrylic acid.

Accordingly the process for producing a polymer in this embodiment of the process comprises the following process steps:
A. dehydrating glycerine in the form of an aqueous glycerine solution to a dehydration product comprising acrolein in the form of an aqueous acrolein solution by means of liquid phase dehydration with at least partial transfer of the aqueous acrolein solution into the gas phase, whereby the weight ratio of acrolein to glycerine in the vapor phase is larger than the weight ratio of acrolein to glycerine in the aqueous acrolein solution by a factor of at least about 2, or at least about 4, at least about 10, or at least about 100;
B. gas phase oxidation the acrolein in the gas phase to obtain a monomer gas comprising acrylic acid;
C. bringing into contact of the monomer gas with a quench means to obtain a quench phase comprising acrylic acid;
D. processing the quench phase to obtain a monomer phase comprising acrylic acid; and
E. polymerizing the monomer phase.

The gas phase oxidation may be carried out within a temperature range from about 200° C. to about 400° C., or from about 250° C. to about 350° C., or from about 280° C. to about 340° C.

The monomer gas may comprise the acrylic acid in an amount within the range from about 5 wt % to about 50 wt %, or from about 10 wt % to about 40 wt %, or from about 15 wt % to about 30 wt % based on the monomer gas.

Water or an organic compound may be used with a boiling point within the range from about 50° C. to about 250° C., or from about 70° C. to about 180° C., or from about 105° C. to about 150° C., or water and this organic compound as quench means in process step c). Organic compounds of this type may be aromatics or alkylated aromatics. The quench means may be generally brought into contact with the monomer gas in a suitable column, such as in counter-current flow. For the case that the quench means comprises at least about 50 wt %, or at least about 70 wt % water, the aqueous quench means charged with acrylic acid may be processed in a further step with a separation means, which may not be very soluble with water. The phase which is richest in acrylic acid may be subjected either to a distillation or to a crystallization or both. The crystallization may be carried out both as layer and as suspension crystallization. Suitable layer crystallization devices are commercially obtainable from Sulzer AG. Suitable suspension crystallization processes may use a crystal generator followed by a washing column. Such devices and processes are commercially obtainable from Niro Prozesstechnologie B.V. Extraction/separating means may be an aromatic compound, or an alkyl aromatic, or toluene. If an organic compound should be used as separating means, this organic compound charged with acrylic acid may likewise be subjected to a distillation and crystallization, or a combination of both. A crystallization suitable for this is disclosed in EP-A-1 015 410.

It is additionally preferred in the process according to the invention that the quench phase comprises the acrylic acid in an amount within the range from 30 to 90 wt. %, preferably within the range from 35 to 85 wt. % and yet more preferably within a range from 45 to 75 wt %, respectively based upon the monomer phase.

The processing of the quench phase may occur at temperatures below the boiling point of acrylic acid. A suitable measure therefore is that the quench phase may already have a temperature of less than about 40° C. by use of a correspondingly cold quench means. The thus-temperature controlled quench phase may then be conducted to an extraction or to a crystallization or both for processing, whereby the temperatures may lie within a range from about −40° C. to about 40° C., or from about −20° C. to about 39° C., or from about −10° C. to about 35° C.

The monomer phase may comprise the acrylic acid in an amount within the range from about 99 wt % to about 99.98 wt %, based upon the monomer phase. Such acrylic acid contents in a monomer phase may appear in particular if the processing occurs by distillation. For the case that the processing occurs by means of extraction and crystallization, the acrylic acid may be present in an amount from about 30 wt % to about 70 wt %, or from about 40 wt % to about 60 wt %, or from about 45 wt % to about 65 wt % in the monomer phase together with water, and the impurities which are different from water and acrylic acid amount to less than about 0.02 wt %, based upon the monomer phase. This aqueous monomer phase has the advantage that it may be used in the aqueous polymerization of the monomer phase without further dilution steps, which may be necessary for the highly concentrated monomer phase.

In addition, the invention relates to a device for production of acrylic acid which comprises the following components connected with each other in fluid-conveying fashion:

1a. a dehydration reactor;
2a. a gas phase oxidation reactor;
3a. a quench unit; and
4a. a processing unit.

In addition, the invention relates to a device for production of polymers, which first comprises the above listed components 1a. to 4a. connected with each other in fluid-conveying fashion and also a polymerization unit 5b.

By fluid-conveying is understood a connection of the individual components, or of their components, by means of pipe systems, or other transport possibilities for gases and liquids, such as tank vehicles.

The dehydration reactor may comprise a compound reservoir suitable for accepting glycerine, followed by a reaction area designed to accept catalyst, in turn followed by a quencher with a line to the gas phase oxidation reactor. These components may be formed from common materials used in the chemical industry which may be inert under the reaction conditions, such as stainless steel or glass. For the case that the reaction area accepts the catalyst as bulk material, it comprises appropriate containers. In another design, the reaction area may also comprise walls that function as catalyst. The quencher is designed as a column, into which water or high-boiling organic solvent may be introduced.

A further aspect of the device may comprise an evaporator after the compound reservoir and before the reaction area. These embodiments may be suitable for gas phase dehydration. For the case that the glycerine with a high salt load from fatty acid saponification is used, the evaporator may comprise a salt separator. If the dehydration occurs in the liquid phase and the acrolein should be evaporated according to the above-described particular embodiment in process step a′, the device may comprise an evaporation device 1a′ arranged between the dehydration reactor 1a. and the gas phase oxidation reactor 2a., which may be connected to the gas phase oxidation reactor 2a. in such a way that the vapor phase obtained in the evaporation device 2a′ is fed into the gas phase oxidation reactor 2a.

According to another embodiment of the device, the reaction area may comprise a lower product outlet that leads into the quencher. This construction has proven itself in liquid phase dehydration. In a further design of the device, the reaction area may be integrated in a cycle, with which reagents and reaction product may be conducted within the cycle.

As gas phase oxidation reactors are considered, all reactors known to the skilled person are suitable for the process according to the invention, which are capable of converting acrolein by gas phase oxidation to acrylic acid. Examples in this context are pipe bundle reactors, or plate reactors, which are cooled with a cooling agent, or with a salt melt. These pipe bundle or plate reactors accommodate a suitable catalyst on the side to which cooling agent may be applied. On the one hand, this may be present as a bulk powder and on the other hand, the surfaces of the pipes or of the plates may be coated with the catalyst.

Quench units may be those types that are known in previous large scale gas phase oxidation of acrolein to acrylic acid. Quench units of this type may be formed as columns or towers and, as for the reactors, can be commercially purchased, for example from Deggendorfer Werft GmbH. Processing units may include all known distillation and crystallization as well as extraction devices known to the skilled person in the art, for large scale acrylic acid synthesis by means of gas phase oxidation of acrolein.

Suitable polymerization units, which may be used in process step E for polymerization of the monomer phase, may be discontinuously operating stirrer vessels or continuously operating systems, such as bulk polymerization devices, extruders, and the like. A comminution and drying follows these polymerization reactors. The thus-obtained superabsorbent precursor may, furthermore, be subjected to a surface- or post-crosslinking. More details concerning this are found in the previously mentioned work from Graham and Buchholz. If the polymers are cross-linked, partially neutralized polyacrylates, reference is made in connection with the exact procedure to the third chapter (page 69 et seq.) in “Modern Superabsorbent Polymer Technology”, F. L. Buchholz and A. T. Graham (Editors) in Wiley-VCH, New York, 1998.

In addition, the process for production of acrylic acid or the process for production of a polymer may occur using the devices described above and more closely illustrated in the figures. In this way, water absorbing polymer structures may be obtained as particularly suitable superabsorbent polymers.

A contribution to the solution of the above-mentioned objects is also made by water-absorbing polymer structures obtainable by radical polymerization of the acrylic acid obtainable by means of the above-described synthetic process in the presence of cross-linkers.

A contribution to the solution of the previously mentioned objects is also made by water-absorbing polymer structures, which are based to at least about 25 wt %, or to at least about 50 wt %, or to at least about 75 wt %, or to at least about 95 wt % based on acrylic acid, whereby at least about 80 wt %, or at least about 90 wt %, or at least about 95 wt % of the acrylic acid monomers used in the production of the water-absorbing polymer structures may be obtained by a synthetic process starting from non-fossil, renewable organic material. These non-fossil, renewable organic materials are in particular materials not generated from petroleum, or coal or brown coal, as well as natural gas. These non-fossil, renewable organic materials are, rather, products of agriculture and forestry, in particular fats and oils from glycerine and fatty acids.

These water-absorbing polymer structures may be obtainable by a process comprising the following process steps:
i) polymerizing the acrylic acid in the presence of a cross-linker to form a polymer gel;
ii) optionally, comminution of the polymer gel;
iii) drying of the polymer gel to obtain water-absorbing polymer structures, and
iv) optionally, surface post-treatment of the water-absorbing polymer structure.

The water-absorbing polymer structures according to the invention may be based to at least about 25 wt %, or to at least about 35 wt %, or to at least about 45 wt % upon natural, biodegradable polymers, or upon carbohydrates such as celluloses or starches.

Polymer structures according to the invention may be fibers, foams, or particles.

Polymer fibers may be dimensioned such that they can be incorporated into or as yarns for textiles and also directly into textiles. The polymer fibers may have a length within the range from about 1 mm to 500 mm, or from about 2 mm to about 500 mm, or from about 5 mm to about 100 mm, and a diameter within the range from about 1 denier to about 200 denier, from about 3 denier to about 100 denier, or from about 5 denier to about 60 denier.

Polymer particles according to the invention may be dimensioned so that they have an average particle size according to ERT 420.2-02 within the range from about 10 μm to about 3000 μm, or from about 20 μm to about 200 μm, or from about 150 μm to about 850 μm. The proportion of particles with a particle size within a range from 300 to 60 μm may be at least about 50 wt %, or at least about 75 wt %.

The water-absorbing polymer structures may have at least one, or both of the following properties:
a CRC value (CRC=Centrifugation Retention Capacity) determined according to ERT 441.2-02 (ERT=Edana Recommended Test Method) of at least about 20 g/g, or at least about 25 g/g, or at least about 30 g/g;
an absorption under a pressure of about 20 g/cm² determined according to ERT 442.2-02 of at least about 15 g/g, or at least about 17.5 g/g, or at least about 20 g/g.

The CRC values and the vales for the absorption under pressure generally do not lie above 150 g/g.

A further contribution to the solution of the above mentioned objects is made by water-absorbing polymer structures, which may be characterized by the following properties:
(β1) the polymer structure is based to at least about 25 wt %, or to at least about 50 wt %, or to at least about 75 wt %, or to at least about 95 wt % on acrylic acid, whereby at least about 80 wt %, or at least about 90 wt %, or at least about 95 wt % of the acrylic acid monomers used in the preparation of the water-absorbing polymer structures has been obtained by a synthesis process which starts from non-fossil, renewable organic material,
(β2) the polymer structure has a biodegradability determined according to the modified Sturm Test according to Appendix V of Guideline 67/548/EWG after 28 days of at least about 25%, or at least about 35%, or at least about 45%, whereby a value of at most about 75 to about 95% as upper limit is generally not exceeded;
(β3) the polymer structure has a CRC value determined according to ERT 441.2-02 of at least about 20 g/g, or at least about 25 g/g, or at least about 29 g/g, whereby a CRC value of about 60 g/g as upper limit is generally not exceeded.

In a further aspect of the polymer structure described in the previous paragraph, said polymer structure has at least properties β1 and β2. All further developments given in this text for the polymer structure are also valid for the polymer structure of this paragraph.

Another contribution to the solution of the above-mentioned objects may be made by water-absorbing polymer structures that may be based to at least about 10 wt %, or at least about 25 wt %, at least about 50 wt %, or at least about 75 wt %, or at least about 80 wt %, based upon the polymer structure, upon acrylic acid and that are characterized by the following properties:
(ε1) the polymer structure has a sustainability factor of at least about 10, or at least about 20, or at least about 50, or at least about 75, or at least about 85, or at least about 95;
(ε2) the polymer structure has a biodegradability determined according to the modified Sturm Test according to Appendix V of Guideline 67/548/EWG after 28 days of at least about 25%, or at least about 35%, or at least about 45%, whereby a value of at most about 75% to about 95% as upper limit is generally not exceeded;
(ε3) the polymer structure has a CRC value determined according to ERT 441.2-02 of at least about 20 g/g, or at least about 25 g/g, or at least about 29 g/g, whereby a CRC value of about 60 g/g as upper limit is generally not exceeded.

In another embodiment of the polymer structure described in the previous section, this polymer structure may have at least properties ε1 and ε2. All further developments given in this text for the polymer structure may be valid for the polymer structure of this paragraph.

In some cases, the above-mentioned upper limits may also be up to about 10% or up to about 20% lower. The polymer structures described in the two previous sections may be based, in addition to the acrylic acid, upon a di- or polysugar. These di- or polysugars may be present as a further component of the polymer structure in an amount of at least about 1 wt %, or at least about 5 wt %, or at least about 15 wt %, based upon the polymer structure, so that the sum of the wt % of the components of the water-absorbing polymer structure is 100 wt %. The sugars of these types may be poly-chain sugars, which may have a number average molecular weight determined by means of gel permeation chromatography and light scattering within the range from about 10,000 g/mol to about 1,000,000 g/mol, or within the range from about 50,000 g/mol to about 500,000 g/mol. These may consist of linear and thus unbranched chains. All sugar compounds known to the skilled person and appearing suitable may be considered as sugars of this type. Thus, for example, celluloses and starches may be mentioned, whereby one or at least two different starches are preferred. Among starches, in turn, amylase-containing starches may be preferred. The amylase content preferably lies within a range from about 10 wt % to about 80 wt %, or within a range from about 20 wt % to about 70 wt %, based upon the starches. The di- or polysugars may have a particle size such that at least about 50 wt %, or at least about 70 wt %, or at least about 85 wt % of the particles are smaller than about 50 μm. The particle size may be determined by means of sieve analysis. Such products are, for example, commercially available under the trade name Eurylon® 7 or Foralys® 380 from the company Roquette, Lestrem, France.

Such water-absorbing polymer structures may be prepared and are thus obtainable preferably by
providing a surface crosslinked water-absorbing polymer;
mixing the surface crosslinked water-absorbing polymer with a di- or polysugar.

The water-absorbing polymer may be based to at least about 50 wt %, or at least about 80 wt %, or at least about 95 wt % upon acrylic acid that comes from the inventive dehydration process used for polymerization partially neutralized and with a crosslinker.

The sustainability factor gives the proportion of the polymer structure which is based upon materials based upon non-fossil, renewable organic materials. A sustainability factor of 100 means that the polymer structure is fully based upon non-fossil, renewable organic materials.

A further contribution to the solution of the above-described objects is provided by a composite comprising the water-absorbing polymer structures according to the invention or water-absorbing polymer structures which are obtainable by radical polymerization of the acrylic acid obtainable by the above-described synthetic process in the presence of cross-linkers. It is preferred that the polymer structures according to the invention and the substrate are firmly bound together. As substrates, sheets made from polymers, such as, for example, made from polyethylene, polypropylene or polyamide, metals, non-wovens, fluff, tissues, wovens, natural or synthetic fibers, or other foams are preferred. It is, furthermore, preferred according to the invention that the polymer structures are comprised in the composite in an amount of at least about 50 wt %, or at least about 70 wt %, or at least about 90 wt %, based upon the total weight of polymer structure and substrate.

In a particularly preferred embodiment of the composite according to the invention, the composite is a sheet-like composite, as described in WO-A-02/056812 as “absorbent material”. The disclosure of WO-A-02/056812, in particular with respect to the exact construction of the composite, the mass per unit area of its components as well as its thickness, is hereby limited as above introduced by reference and represents a part of the disclosure of the present invention.

A further contribution to the solution of the above-mentioned objects is provided by a process for producing a composite, whereby the water-absorbing polymer structures according to the invention, or the water-absorbing polymers, which can be obtained by radical polymerization of the acrylic acid obtainable by the above-described synthetic process in the presence of cross-linkers, and a substrate and optionally an additive are brought into contact with each other. Substrates that may be used are those substrates that have already been mentioned in connection with the composite according to the invention.

A contribution to the solution of the above-mentioned objects may also be provided by a composite obtainable by the above-described process.

A further contribution to the solution of the above-mentioned objects may be delivered by chemical products comprising the water-absorbing polymer structures according to the invention or a composite according to the invention. Examples of chemical products include foams, moulded bodies, fibers, sheets, films, cables, sealing materials, liquid-absorbing hygiene articles, in particular diapers and sanitary napkins, carriers for plant or fungus growth-regulating agents or plant protection agents, additives for construction materials, packaging materials, or soil additives. Chemical products such as hygiene articles may comprise an upper layer, a lower layer, and an intermediate layer arranged between the upper layer and the lower layer, which comprises water-absorbing polymer structures according to the invention.

In addition, the invention relates to a process for the production of acrolein that may be characterized by the herein described process for dehydration of glycerine to a dehydration product comprising acrolein and the herein described preferred embodiments of this dehydration.

The invention further relates to fibers, sheets, adhesives, cosmetics, moulding materials, textile and leather additives, flocculants, coatings or varnishes based upon acrylic acid which is obtainable according to a process according to the invention, or their derivatives or salts. Derivatives of acrylic acid may include its esters, or its alkyl esters, or its C₁ to C₁₀, or C₂ to C₅, or C₃ to C₄ alkyl esters. Salts may include the alkali or alkaline earth as well as the ammonia salts of acrylic acid.

The invention further relates to the use of an acrylic acid which has been obtained by a process according to the invention, or derivatives or salts thereof in fibers, sheets, adhesives, cosmetics, moulding materials, textile and leather additives, flocculants, coatings, or varnishes.

The invention is now illustrated by means of non-limiting figures and examples.

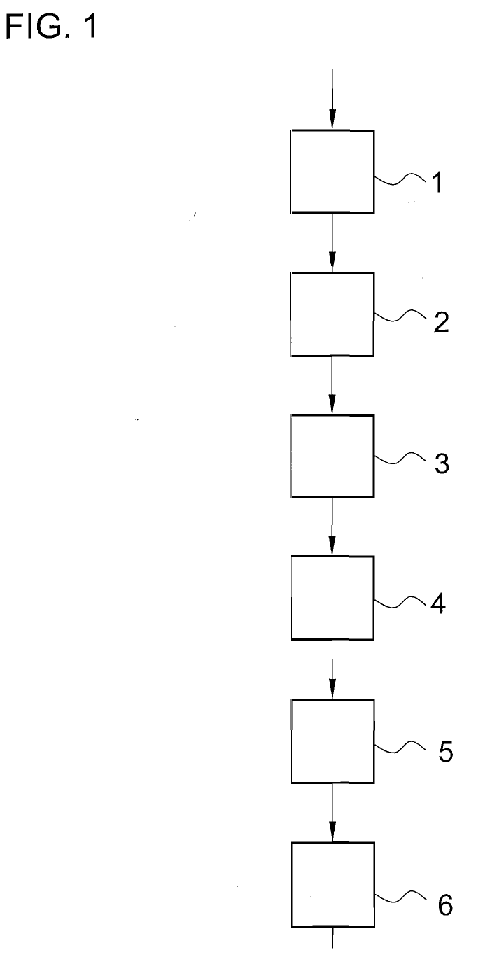

FIG. 1 shows a schematic of the individual stages and steps of the process according to the invention and of the device according to the invention.

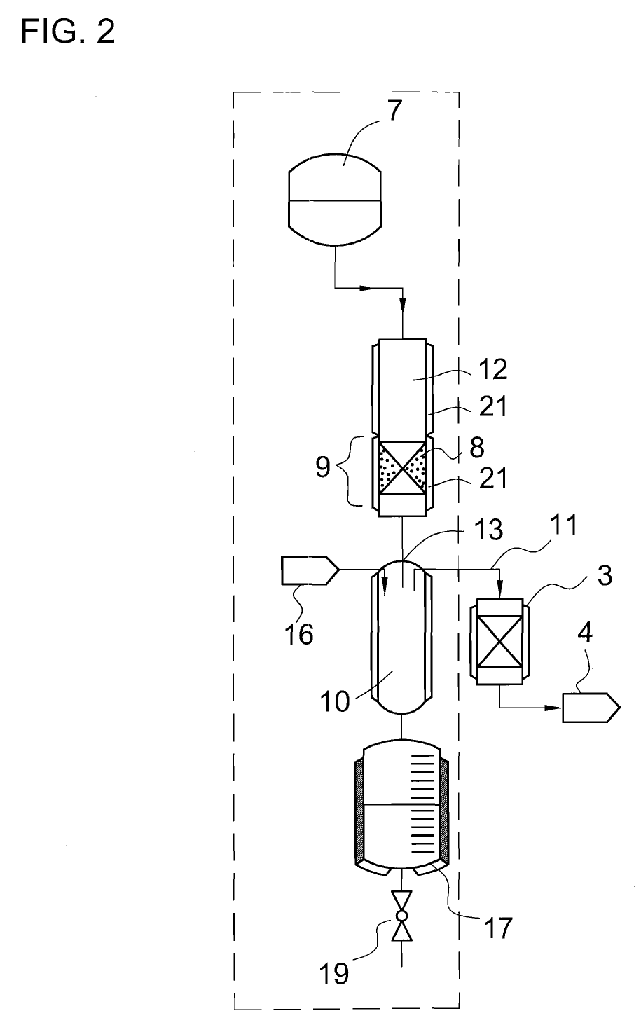

FIG. 2 shows a gas phase dehydration unit.

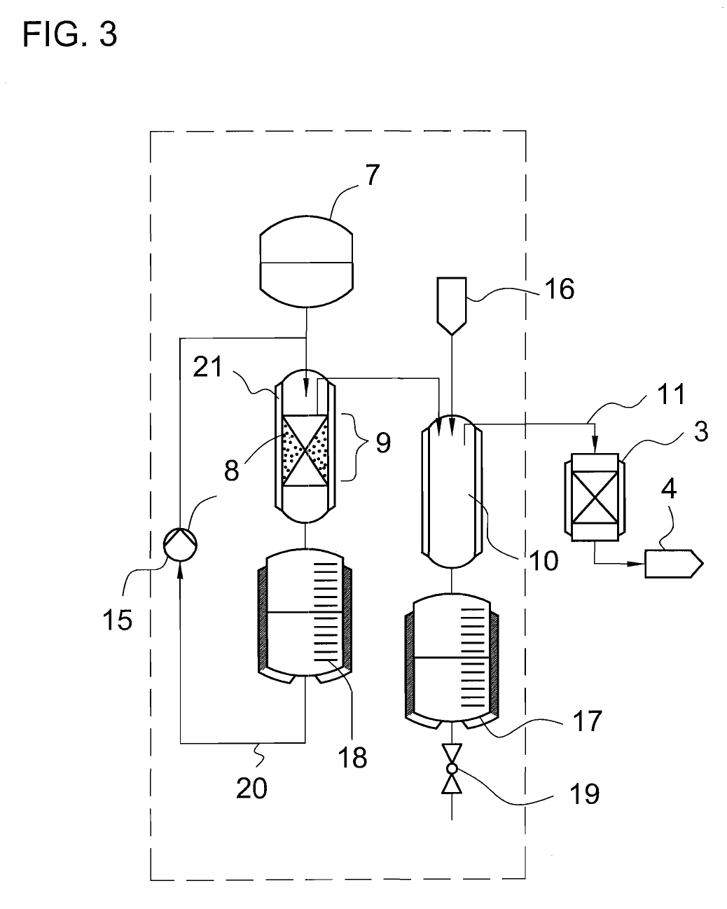

FIG. 3 shows a liquid phase dehydration unit.

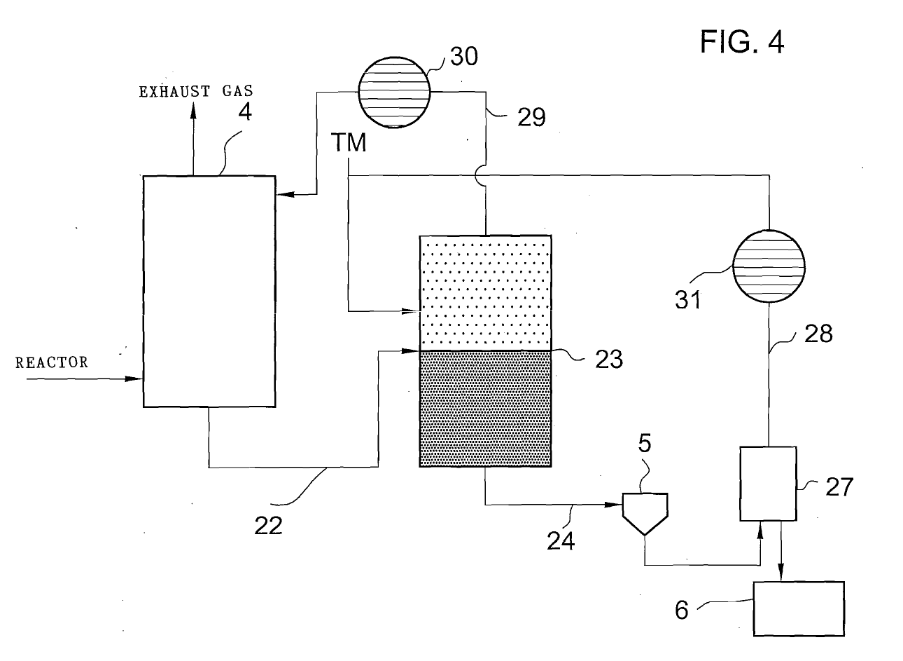

FIG. 4 shows a processing unit.

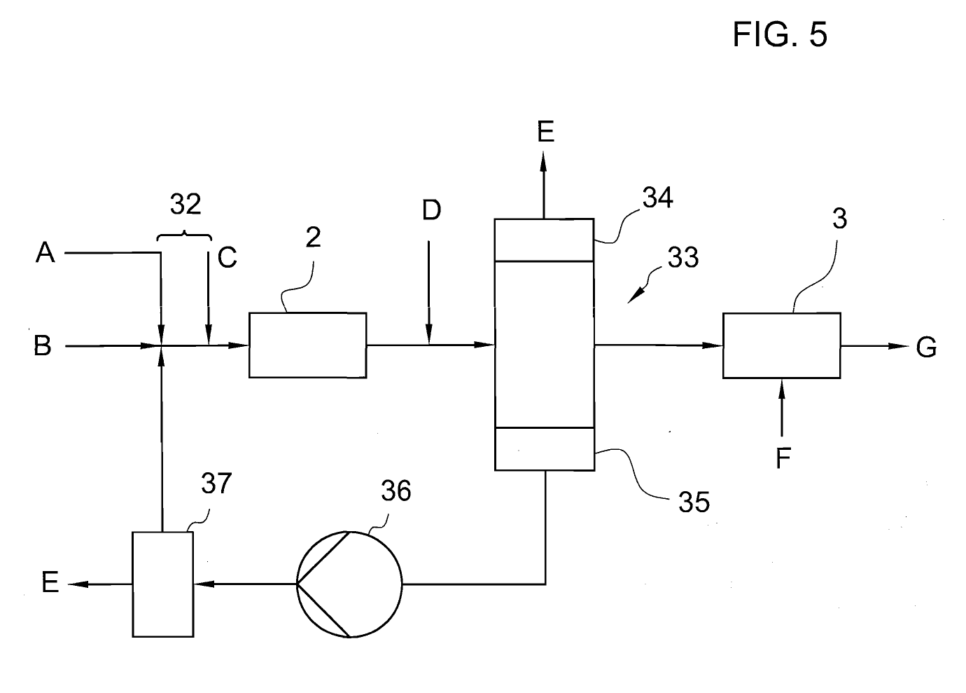

FIG. 5 shows a further embodiment of the liquid phase dehydration unit.

In FIG. 1, firstly the oils or fats are introduced into a saponifier 1, where an alkaline saponification with bases or alkali alcoholates occurs. The glycerine produced in the saponifier by generally known purification steps such as salting out and distillation is then conduced to a dehydration unit with a dehydration reactor 2 (in order to produce acrolein from the glycerine). The thus-produced acrolein is then conducted in a next step to a gas phase reaction reactor 3, in which it is converted by a gas phase oxidation reaction into acrylic acid. The acrylic acid-comprising gas from reactor 3 goes to a quench unit 4, in which the acrylic acid-comprising gas is converted into the liquid phase by bringing it into contact with a quench means. The liquid mixture of quench means and acrylic acid is conducted to a processing unit 5 following the quench unit 4. There, the acrylic acid is purified to pure acrylic acid (at least 99.98% acrylic acid) either by crystallization or distillation or by a combination of these two steps or by extraction, or a combination of extraction and crystallization, or a combination of extraction and distillation, or a combination of extraction-distillation and distillation, the acrylic acid being present as pure acrylic acid itself or in an aqueous phase. The thus-obtained acrylic acid is then conducted to a polymerization unit 6. The polymer obtained in the polymerization unit 6 can be processed according to the subsequent use. A further processing unit, for example a diaper machine or a machine for production of binding and wound material can follow after the polymerization unit 6.

FIG. 2 shows a gas phase dehydration unit, in which a compound reservoir 7 is connected to an evaporator 12. The evaporator 12 is connected upstream to a reaction area 9. The pressure reaction area 9 comprises catalyst 8. Both the evaporator 12 and the reaction area 9 comprise heating elements 21, with which the temperatures necessary for evaporation and dehydration of glycerine can be achieved. The reaction area 9 is connected to a quencher 10 via a lower outlet 13. The quencher 10 receives, in its upper area, in addition to the lower product outlet 13, a quench liquid feed 16. In the lower area of the quencher 10, a trapping reservoir for side-products 17 is arranged, which can be emptied by means of exit valve 19. The upper area of the quencher 10 further receives an exit line 11, which conducts the acrolein-comprising dehydration product to gas phase reaction reactor 3. Quench unit 4 follows these.

FIG. 3 shows a liquid phase dehydration device, in which glycerine is placed in a compound reservoir 7, which is connected with the upper area of a pressure reaction area 9, which in turn comprises catalysts 8. The reaction area 9 is temperature-controlled by means of a heating element 21. In the lower area of reaction area 9, a buffer reservoir 18 is arranged, which forms a cycle with the reaction area by means of a pump, and via which glycerine and acrolein already situated in the reaction area 9 and in the buffer reservoir can be conducted in the cycle. In the upper area of the reaction area 9 is situated a transfer line to a quencher 10, which leads into the upper area of the quencher 10 together with a quench liquid feed 16. Also in the upper area of the quencher 10 is situated an exit line 11 to the gas phase reaction reactor 3, followed in turn by the quench unit 4. In the lower area of the reaction area 9, a trapping reservoir for side-products 17 equipped with an exit valve 9 has an opening.

In FIG. 4 the gas coming from the gas phase reaction reactor 3 is introduced into the quench unit 4, in which it is brought into contact with water and absorbed, whereby as absorption liquid an aqueous acrylic acid solution is obtained, in which likewise a part of the side-products arising from the previous synthesis steps is comprised. This aqueous acrylic acid solution is conducted via a feed line 22 to an extraction unit 23. At the latest in the extraction unit 23, the acrylic acid solution is brought into contact with toluene as a separating agent. After a careful combination in the extraction unit 23, a phase separation occurs to form an upper phase based substantially on water, and a lower phase based substantially on toluene as a separating agent. The phase based substantially on the separating agent is conducted via feed line 24 to the crystallizer 25, which is preferably a scratch cooler. The crystal suspension comprised in crystallizer 25 is then conducted by means of a suspension conduit 26 to a wash column 27, in which a separation of the acrylic acid crystals occurs, in the course of which a mother liquor comprising the separating agent is retained. The mother liquor obtained in the wash column 27 after the separation of the acrylic acid crystals is preferably at least partially conducted back via the mother liquor conduit 28 into the extraction unit 23. It is further preferred that the upper phase based substantially on water comprised in the extraction unit 23 is conducted back to the quench unit 4 via quench conduit 29. In a preferred design of the process according to the invention and of the device according to the invention, acrylic acid still comprised in the composition can be separated by crystallization from the composition conducted in the quench conduit 29 by means of a further purification device 30, which is, for example, a suspension crystallization device or a layer crystallizer. It is, furthermore, preferred in another embodiment of the process according to the invention and of the device according to the invention that the composition conducted in the mother liquor conduit 28 (mother liquor separated during the crystallization) is separated from further impurities before its being conducted back by means of a separating unit 31. This separating unit is preferably likewise a suspension crystallization device or a layer crystallizer or also a filter.

In FIG. 5 is shown schematically that optionally in a pre-mixer 32, via lines A to C, water, glycerine and, in so far as necessary, catalyst, for example an inorganic acid such as phosphoric acid, are combined and conducted to the dehydration reactor 2 formed as a stainless steel pressure reactor. The dehydration of glycerine to acrolein in the liquid phase occurs here. When using a soluble and thus homogeneously distributable catalyst, such as an inorganic acid, it is advantageous to at least partially neutralize this by the addition of a base such as NaOH, mostly as a solution, via line D. This measure contributes to the prevention of further reactions which can lead to undesired side products. Following this is a distillation device 33 which comprises in the upper section a volatiles area 34 and in the lower section a high-boilers area 35, whereby the acrolein-comprising phase from the dehydration is introduced between the two areas 34 and 35. In the volatiles area 34, the components with lower boiling points than acrolein would come out guided by exit line E and the still remaining acrolein and the high-boiling components guided in the reflux. In the high-boiler area 35 representing the distiller bottom, the high-boilers are concentrated. It is here advantageous to conduct the bottom product in a cycle. From the high-boiler area, the components with higher boiling points than acrolein are discharged and conducted via a high-boiler pump to a high boiler separator 37. The high-boiler separator is advantageously equipped with a membrane. After passing through the high-boiler separator 37, the mixture, comprising predominantly water and glycerine, freed from the high-boilers, is conducted back to the pre-mixer 32 and is thus available again for dehydration. In this way an efficient dehydration of glycerine to acrolein can be achieved by the cyclical route, in which the thus-obtained acrolein from the distiller 33 in sufficient purity for a long-lasting operation of the gas phase oxidation is conducted to the gas phase reaction reactor 3 for oxidation with air conducted in through feed line F to acrolein conducted away via exit line G.

### EXAMPLES

The following examples and preproducts for the examples are provided to illustrate the invention and do not limit the scope of the claims. Unless otherwise stated, all parts and percentages are by weight.

### Example 1

### Dehydration in the Gas Phase

In a gas phase dehydration device described in FIG. 2 (enclosed within the dashed line), a catalyst is provided in reactor area 9, the catalyst being obtained from 100 parts by weight of Rosenthal balls (alpha-Al₂O₃) with a diameter of 3 mm by mixing with 25 parts by weight of a 20 wt % phosphoric acid for 1 hour and then separated from excess water by rotary evaporation at 80° C. (H₀ value between −5.6 and −3). The glycerine evaporated in an evaporator at 295° C. is conveyed over 100 ml of this catalyst in a steel pipe in the reaction area as a 20 wt % aqueous solution with a pump with 40 ml/h at a temperature of 270° C. The acrolein-comprising reaction mixture was brought into contact with water as quench means in the quencher and the thus-obtained aqueous mixture conducted to the gas phase oxidation in a conventional reactor for gas phase oxidation of acrolein to acrylic acid.

### Example 2

### Liquid Phase Dehydration

In a device according to FIG. 3, a catalyst as described in Example 1 was used for liquid phase dehydration (enclosed within the dashed line), whereby in the place of the Rosenthal balls a silicon dioxide carrier was used (H₀ value between 2 and −3). The reaction temperature was 240° C. Water was used as quench means. Following the acrolein synthesis, a gas phase oxidation in a conventional gas phase oxidation reactor followed, as in Example 1, followed by an absorption in water in a quench unit.

The acrylic acid-water mixtures obtained in examples 1 and 2 were combined in a glass separating funnel temperature-controlled to 0° C. with 0.5 parts of their volume of toluene. The mixture was shaken vigorously and left to stand for 60 minutes in order to enable a phase separation. The two thus-arising phases were separated. The toluene-comprising phase was subjected to an azeotropic distillation and the thus-obtained acrylic acid distilled before its use in polymerization.

PTA could not be identified by gas chromatography as an impurity in the acrylic acid obtained from acrolein obtained either by gas phase dehydration or by liquid phase dehydration followed by gas phase oxidation.

### Example 3

### Polymerization

Dissolved oxygen was removed from a monomer solution consisting of 280 g of the above-obtained acrylic acid, which was neutralized to 70 mol. % with sodium hydroxide, 466.8 g water, 1.4 g polyethylene glycol-300-diacrylate, and 1.68 g allyloxypolyethyleneglycol acrylic acid ester by flushing with nitrogen and the monomer solution cooled to the start temperature of 4° C. After reaching the start temperature, the initiator solution (0.1 g 2,2-azobis-2-amidinpropane dihydrochloride in 10 g H₂O, 0.3 g sodium peroxydisulfate in 10 g H₂O, 0.07 g 30% hydrogen peroxide solution in 1 g H₂O, and 0.015 g ascorbic acid in 2 g H₂O) was added. After the end temperature of approximately 100° C. was reached, the gel formed was comminuted and dried for 90 minutes at 150° C. The dried polymer was coarsely chopped, ground, and sieved to a powder with a particle size of 150 to 850 μm.

For post-crosslinking, 100 g of the above-obtained powder was combined with a solution of 1 g 1,3-dioxalan-2-one, 3 g water, and 0.5 g aluminium sulphate-18-hydrate and then heated for 40 minutes in an oven set to 180° C.

### Example 4

### Preparation of a Biodegradable Polymer

The post-crosslinked polymer obtained in Example 3 was mixed under dry conditions with a water-soluble wheat starch (the product Foralys® 380 from the company Roquette, Lestrem, France) in the weight ratio polymer:starch of 4:1 and then homogenized for 45 minutes in a roll mixer type BTR 10 of the company Frobel GmbH, Germany.

The product had a biodegradability according to the modified Sturm test after 28 days of 39% and a CRC value of 29.9 g/g. The sustainability factor was about 0.99.

### REFERENCE CHARACTERS

1 saponifier
2 dehydration reactor
3 gas phase reaction reactor
4 quench unit
5 processing unit
6 polymerization unit
7 compound reservoir
8 catalyst
9 reaction area
10 quencher
11 exit line
12 evaporator
13 lower product outlet
14 upper product outlet
15 pump
16 quench liquid feed
17 trapping reservoir for side products
18 buffer reservoir
19 outlet valve
20 cycle
21 heating element
22 feed line
23 extraction unit
24 feed line
25 crystallizer
26 suspension conduit
27 wash column
28 mother liquor conduit
29 quench conduit
30 purification device
31 separating unit
32 pre-mixer
33 distiller
34 volatiles area
35 high-boilers area
36 high-boilers pump
37 high-boilers separator

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

1. A process for production of acrylic acid, comprising at least the following steps:
a. dehydrating glycerine to a dehydration product comprising acrolein;
b. gas phase oxidation of the dehydration product comprising acrolein to obtain a monomer gas comprising an acrylic acid;
c. bringing into contact the monomer gas with a quench means to obtain a quench phase comprising acrylic acid; and
d. processing of the quench phase comprising acrylic acid to obtain a monomer phase comprising acrylic acid.

2. The process according to claim 1 further including the step of:
E. radical polymerization of the monomer phase.

3. The process according to claim 2, wherein the polymer is a water-absorbing polymer structure.

4. The process according to claim 3, wherein the water-absorbing polymer structure is obtainable by a process comprising the following process steps:
i) polymerizing the acrylic acid in the presence of a crosslinker to form a polymer gel;
ii) optionally comminuting the polymer gel;
iii) drying of the comminuted polymer gel to obtain a water-absorbing polymer structure; and
iv) optionally surface post-crosslinking of the water-absorbing polymer structure.

5. The process according to claim 2, wherein the acrylic acid is present to at least about 20 mol %, based on the monomer, as a salt.

6. The process according to claim 1, wherein the glycerine is obtained from the saponification of fats.

7. The process according to claim 1, wherein glycerine is generated in the generation of liquid fuels from natural raw materials.

8. The process according to claim 1, wherein the dehydration occurs at least partially in a liquid phase.

9. The process according to claim 1, wherein the dehydration occurs at least partially in a gas phase.

10. The process according to claim 1, wherein the glycerine is used in aqueous phase.

11. The process according to claim 1, wherein a dehydration catalyst is used.

12. The process according to claim 11, wherein the dehydration catalyst is in contact with a carrier x.

13. The process according to claim 12, wherein the carrier x is a solid with a total pore volume according to DIN 66133 within a range from about 0.1 to about 1.5 ml/g.

14. The process according to claim 11 wherein the dehydration catalyst is an inorganic acid.

15-23. (canceled)

24. A device for production of acrylic acid comprising being connected with each other in fluid-conveying fashion:
1a. a dehydration reactor,
2a. a gas phase oxidation reactor,
3a. a quench unit, and
4a. a processing unit.

25. The device of claim 24 further comprising
5a. a polymerization unit.

26. The device according to claim 24, wherein the dehydration reactor comprises
a compound reservoir, followed by
a reaction area accepting a catalyst, followed by
a quencher with a line to the gas phase oxidation reactor.

27. The device according to claim 26, wherein an evaporator is provided after the compound reservoir and before the reaction area.

28. (canceled)

29. The device according to claim 27, wherein the reaction area comprises an upper product outlet which leads into the quencher.

30-32. (canceled)

33. A water-absorbing polymer structure based to at least about 25 wt. % upon optionally partially neutralized acrylic acid, wherein the water-absorbing polymer structures are characterized by a sustainability factor of at least about 80%.

34. The water-absorbing polymer structure according to claim 33, wherein the polymer structures are based to at least about 25 wt. %, based upon the total weight of the water-absorbing polymer structure, upon natural, biodegradable polymers.

35. A water-absorbing polymer structure according to claim 33 characterized by the following properties:
(β1) the polymer structure is based to at least about 25 wt. % on acrylic acid, whereby at least about 80 wt. % of the acrylic acid monomers used in the preparation of the water-absorbing polymer structures has been obtained by a synthesis process which starts from non-fossil, renewable organic material,
(β2) the polymer structure has a biodegradability determined according to the modified Sturm Test according to Appendix V of Guideline 67/548/EWG after 28 days of at least about 25%; and
(β3) the polymer structure has a CRC value determined according to ERT 441.2-02 of at least about 20 g/g.

36. A water-absorbing polymer structure according to claim 33 based to at least about 10 wt. %, based upon the polymer structure, upon acrylic acid and which is characterized by the following properties:
(ε1) the polymer structure has a sustainability factor of at least about 10;
(ε2) the polymer structure has a biodegradability determined according to the modified Sturm Test according to Appendix V of Guideline 67/548/EWG after 28 days of at least about 25%; and
(ε3) the polymer structure has a CRC value determined according to ERT 441.2-02 of at least about 20 g/g.

37. A composite comprising a water-absorbing polymer structure according to claim 33 and a substrate.

38-39. (canceled)

40. A hygiene article comprising an upper sheet, a lower sheet and an intermediate sheet arranged between the upper sheet and the lower sheet, which comprises water-absorbing polymer structures according to claim 33.

41-42. (canceled)
